(19) United States
(12) Patent Application Publication
(10) Pub. No.: US 2025/0361677 A1
(43) Pub. Date: Nov. 27, 2025

(54) Method for controlling a process in making pulp, paper, or board

(71) Applicant: Kemira Oyj, Helsinki (FI)

(72) Inventor: Marko KOLARI, Helsinki (FI)

(21) Appl. No.: 18/874,116

(22) PCT Filed: Jun. 19, 2023
(86) PCT No.: PCT/FI2023/050364

§ 371 Date: Dec. 12, 2024

(30) Foreign Application Priority Data
Jun. 23, 2022 (FI) 20225576

(51) Int. Cl. D21G 9/00; D21C 3/22

(52) U.S. Cl. CPC D21G 9/0018; D21C 3/228

(57) ABSTRACT

A method, apparatus, and computer program control a process in making pulp, paper, or board automatically controlling by a rules engine a dosing of a chemical agent in the process into a fibrous suspension. The rules engine receives a plurality of measurements relating to the process and maintains a plurality of rules sets, including an active rules set. Each rules set defines how the dosing depends on the measurements. The rules engine controls the dosing of the chemical agent based on the measurements and the active rules set, logs at least some of the measurements and actualised dosing of the chemical agent, and receives at least one rules set candidate. The dosing of the chemical agent is simulated with the received at least one rules set candidate based on the earlier measurements; and simulation results are output for optimising performance of the chemical agent in the process.

### TECHNICAL FIELD

The present disclosure generally relates to a method for controlling a process in making pulp, paper, or board.

### BACKGROUND

This section illustrates useful background information without admission of any technique described herein representative of the state of the art.

Making pulp, paper, and board each are based on natural polymers typically obtained from wood. In each case, pulp is either manufactured or used to form a fibrous water suspension of wood-based fibres, mostly of cellulose and hemicellulose. Mechanical pulps may further comprise significant amounts of lignin.

As a natural product, the fibrous suspension is fermentable by microbes and therefore the process is exposed to quality disturbances through undesired microbe action. Biocidal chemicals may be dosed to process to control microbial activity. Moreover, various chemical additives such as retention aids, strength agents, defoamers, fixing agents, dyes, hydrophobisation of paper with sizing agents, dispersion agents, bleaching agents, pH control agents, and fillers may be dosed into the fibrous suspension for controlling various properties of the ready pulp, paper or board. Depending on implementation, dosing into the fibrous suspension may be arranged directly into the fibrous suspension or via an intermediate substance, such as water. Regardless of the chemical agent in question, it is generally desirable to optimise performance of the chemical agent in the process to avoid excess use while attaining required targets in process conditions or in final product quality, such as opacity, brightness, printability, colour, water or grease resistance, tensile strength, square mass uniformity, hygiene of board, process productivity (low number of breaks), low microbial activity in process, stable process pH and conductivity, low amount of slime or other deposits on machine surfaces, etc. with correct dosing.

Circumstances in the process are constantly changing and therefore it is well understood that adaptive control of the dosing of chemicals is beneficial. However, for a good performance the adaptive dosing control of chemicals needs to take in account plurality of concurrent changes, some of which may have opposite effects. It is further difficult or impossible to acquire online measurements of some desired properties of the produced material. Therefore, it is particularly desirable to enable rapid and accurate adjustment of the control process of chemical agent dosing to allow subsequent correction of identified shortcomings in the dosing control.

### SUMMARY

The appended claims define the scope of protection. Any examples and technical descriptions of apparatuses, products and/or methods in the description and/or drawings not covered by the claims are presented not as embodiments of the invention but as background art or examples useful for understanding the invention.

According to a first example aspect there is provided a method as defined by appended claim 1.

According to a second example aspect there is provided an apparatus as defined by appended claim 14. The apparatus may comprise a communication interface configured to receive measurements and at least one processor configured to cause the apparatus to perform the method of the first example aspect.

According to a third example aspect there is provided computer program comprising computer executable program code which when executed by at least one processor causes a computer at least to perform the method of the first example aspect.

According to a fourth example aspect there is provided a computer program product comprising a non-transitory computer readable medium having the computer program of the third example aspect stored thereon.

According to a fifth example aspect there is provided an apparatus comprising means for performing the method of any preceding aspect.

Any foregoing memory medium may comprise a digital data storage such as a data disc or diskette; optical storage; magnetic storage; holographic storage; opto-magnetic storage; phase-change memory; resistive random-access memory; magnetic random-access memory; solid-electrolyte memory; ferroelectric random-access memory; organic memory; or polymer memory. The memory medium may be formed into a device without other substantial functions than storing memory or it may be formed as part of a device with other functions, including but not limited to a memory of a computer; a chip set; and a sub assembly of an electronic device.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments may apply to other example aspects as well.

### BRIEF DESCRIPTION OF THE FIGURES

Some example embodiments will be described with reference to the accompanying figures, in which:

FIG. 1 schematically shows process equipment according to an example embodiment;

FIG. 2 shows a block diagram of an apparatus according to an example embodiment; and

FIGS. 3a and 3b show a flow chart according to an example embodiment.

### DETAILED DESCRIPTION

In the following description, like reference signs denote like elements or steps.

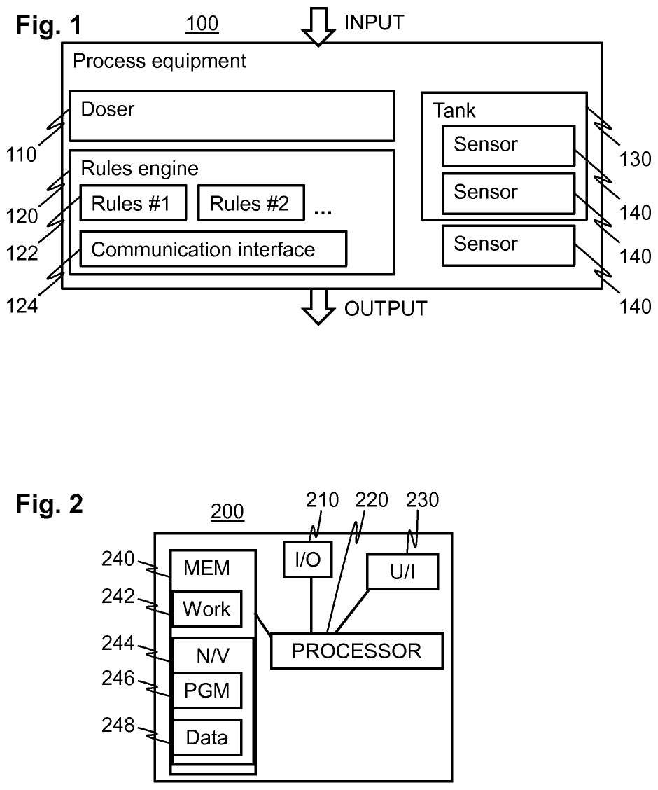

FIG. 1 schematically process equipment 100 in which a pulp, board, or paper making process is performed, including a chemical agent doser 110 configured to dose a chemical agent to a fibrous suspension. The process equipment 110 further comprises a rules engine 120, a tank 130, and a plurality of sensors 140. The process rules engine 120 comprises a rules set 122 and a communication interface 124. The rules set 122 comprises rules according to which the doser 110 is controlled to dose a chemical agent. The rules engine inputs measurements and controls the dosing according to the rules set. In an example embodiment, the rules engine 120 is implemented by a computing entity such as a dedicated server; a virtualised server; cloud computing; or a computer with further tasks. In an example embodiment, the rules engine comprises a logics component, and one or more rules sets. The logics component is configured to control operation of the rules engine 120 according to an active rules set that is currently in use by the rules engine 120.

As drawn in FIG. 1, the rules engine 120 is capable of storing a plurality of rules sets 122 and using one of the rules sets 122 as the active rules set defining that how the rules engine 120 controls the dosing of the chemical agent.

In an example embodiment, the dosing of the chemical agent comprises batch dosing. In an example embodiment, the dosing of the chemical agent comprises continuous dosing. Moreover, the pulp, board, or paper making process itself may be or comprise a batch process and/or a continuous process.

The dosing may be applied at a dosing point in any one or more location in the pulp, paper or board making process, such as in a pipeline, in a tank, in a large storage tower, in a fractionator, in a thickener, in headbox, and/or in dewatering section. In controlling the dosing, the amount and/or timing of the dosing may be adjusted. Moreover, or alternatively, distribution of the dosing between two or more dosing locations may be adjusted.

In some example embodiments the dosing is controlled by adjusting that how much a given chemical agent is administered to the fibrous solution. In an example embodiment, the dosing can further or alternatively be adjusted by changing composition or concentration of the chemical agent. For example, an increased effect may be attained by either increasing the dosing or dosing in some more effective chemical(s). One or more rules in the rules set may define that how the dosing is performed for different desired effect levels. One or more rules in the rules set may define that how the dosing is performed for different paper or board grades in production.

The process may constitute an entire pulp, paper, or board making process or a portion thereof. In an example embodiment, the process comprises buffering the fibrous suspension. The buffering may be performed, for example, using an intermediate storage comprised by the process equipment. In FIG. 1, the storage is a tank 130. In an example embodiment, the storage is a storage tower.

The sensors 140 may comprise two or more sensors that measure same quantities, optionally with same scale. The sensors may comprise a plurality of different sensors measuring different properties or different scales of same properties.

The sensors 140 may further comprise one or more state sensors configured to indicate respective states of process devices such as pumps, motors, or valves. For example, a storage mixing pump or shredder motor state may be provided by a state sensor. In an example embodiment, the state sensor is a piece of hardware. In another example embodiment, the state sensor is implemented by software. For example, the state sensor can be formed by a process automation system that outputs a state of a process device or an actuator that operates a process device.

In an example embodiment, the pulp, board, or paper making process in question comprises shredded storing paper mill waste suspension, i.e., broke, in a storage. The suspension can be a water suspension of a relatively low dry content. Depending on implementation, the suspension is stored in a tank with intermittent or continuous supply. In an example embodiment, the tank provides an intermittent or continuous output to downstream equipment. In sake of example, let us assume that suspension is stored in a cylindrical pass-through storage with an input at top and an output connection at bottom. Depending on throughput rate, an average transfer time of the suspension is, e.g., 2 to 20 hours. However, dead zones can appear near the output end where previously stored suspension is replaced at far slower rate than the average. In one example, dosing of a biocide agent is adapted such that the microbial fermentation activity in the tower is as low as possible. The microbial fermentation activity may depend on a plurality of factors, including but not limited to dry weight content in the suspension, homogeneity of the suspension, storage mixing efficacy, inventory of suspension, temperature, time since previous washing of process equipment, time since last time emptying the storage tank completely, and microbial activity in other portions of the production process. In this context, term inventory refers to a production time that can be satisfied by the material in the storage, here by the fibrous suspension.

In a simplest case, dosing of a chemical is proportional to one parameter, such as inflow of the suspension. A slightly more advanced control combines more parameters, such as storage tower inventory and pH of the suspension. Assuming the chemical agent being a biocide, bigger inventory means more time for microbial growth, and pH drop is an indicator of increased microbial fermentation and both of these parameters can be used for adjusting dosing rate of the biocide. However, there may be a plurality of further parameters that have influence on the process and that should be accounted for in the dosing of the chemical agent. It is laborious and difficult to define process automation rules to control the dosing in such cases, particularly for every possible scenario. In practice, it may be impossible to anticipate all the scenarios, particularly in the start of a chemical agent dosing. It may be simply observed after the fact that the dosing has failed as either chemical consumption has excessively increased, or the process has failed resulting in an inferior product.

A normal process automation is relatively easy to adjust by changing coefficients such as offsets and multipliers applied to each measurement in a formula that defines the dosing. However, in line with the aforementioned difficulties in anticipating scenarios in the process development, it is also extremely difficult to anticipate even which parameters should be used to control the dosing. Maybe some new measurements should be added, or new combination parameters be formed that combine a plurality of measurements with each other and/or with time. For example, it may be meaningful for the dosing of the chemical how long a time has lapsed since the more recent of two events that are a) a latest washing of the process equipment and b) a time when a surface level of the suspension has reached one or more particular levels in a storage. Or, time, temperature, pH, previously measured microbiological activity, and some dwell time-based parameter of the suspension may be meaningful for determining the dosing. In such a case, the dwell time based parameter might be defined for a 10% volume portion of the suspension that has remained for a longest time in the storage, mean, average, or any combination of such derived parameters. Also following a parameter in time, such as brightness, pH, conductivity, or inventory, and identifying rapid changes by comparing latest values to past average values, may be meaningful for determining the dosing. Such combined parameters could be formed with any functions, including but not limited to addition, subtraction, multiplication, powers, variance, standard deviation, logarithms, look-up table based functions, and any combinations of these.

In an example embodiment, different parameters are used in controlling the dosing in different circumstances. For example, the rules set 122 in use may comprise ranges or scenarios defined for dynamically altering the control of the dosing while using one particular rules set 122. In an example embodiment, the rules set may define one or more abort rules configured to change operation of the active rules set. The operation may be changed by performing an additional starting or stopping of the dosing. In an example embodiment, the dosing is aborted, for example, when reaching a set limit for maximum dosing quantity in a given time. In an example embodiment, a possible trigger for abort dosing is a measurement value that reaches a set limit indicative for reaching a sufficient dosing response, or for example reaching a set limit for low flow situation in a given process line. In an example embodiment, in case of a batch dosing, an additional dosing is triggered via an abort rule, for example, when a process parameter reaches a set threshold limit value indicative for rapid need for an additional dosing batch. For example, the parameter can be a low process pH, a low oxidative status of the process (e.g., oxidation reduction potential ORP or redox), high conductivity, big inventory, a different production grade starting, or a set time limit reached for dwell time in a storage tank.

It is quite impractical to empirically optimise the dosing by gradually changing the process automation. By the time one rare process scenario would become decently addressed, new ones could arise. It is thus desirable to form, adapt, and test various rules sets 122 based on earlier measurements made relating to the process by simulating with earlier measurements how the dosing would have in response to changes in the process. The simulating optionally further estimates one or more properties in the product. Alternatively, the simulation facilitates expert evaluation of suitable changes in the active rules set such that undesired quality issues or excess chemical use could be avoided. Advantageously, the simulation may enable illustrating how one or more alternative new rules sets would have controlled the dosing with real historical process conditions. An expert can thus simulate the dosing control with any rules sets as desired with past measurements to verify and then adopt a desired rules set into production as a new active rules set. Verifying of process control improvement can thus be significantly accelerated. In result, manufacturing of pulp, paper, or board can be improved far sooner and more agile than before. Quality problems, production breaks, and/or excess chemical agent use can thus be reduced.

In an example embodiment, the chemical agent comprises a biocidal chemical, a defoamer, a retention chemical, a retention polymer, a strength chemical, a fixing agent, a dye, a hydrophobisation agent, a dispersion agent, a bleaching agent, and/or a pH control agent.

In an example embodiment, the measurements relating to the process comprise any of the following: a state of an actuator; flow rate of a process material; temperature; time; filling rate of a storage; process break time; a process condition; a property of the product of the process, such as moisture of manufactured paper or paper board, brightness, board strength, opacity variation, track break frequency; chemical consumption; air content, speed of process flow, and/or electrical conductivity.

In an example embodiment, the rules engine 120 controls at least one or two dosing points. In an example embodiment, the rules engine 120 controls at most 10, 8, 6, or 5 dosing points. In an example embodiment, the rules engine 120 is coupled to the doser 110. In another example embodiment, another controller resides between the rules engine 120 and the doser 110 or a plurality of dosers 110. The controller may be, e.g., a programmable logic controller, PLC. In an example embodiment, the rules engine 120 is capable of operating off-line in case of no network connectivity exists. In another example embodiment, the controller is configured to implement controlling of the process in off-line situations.

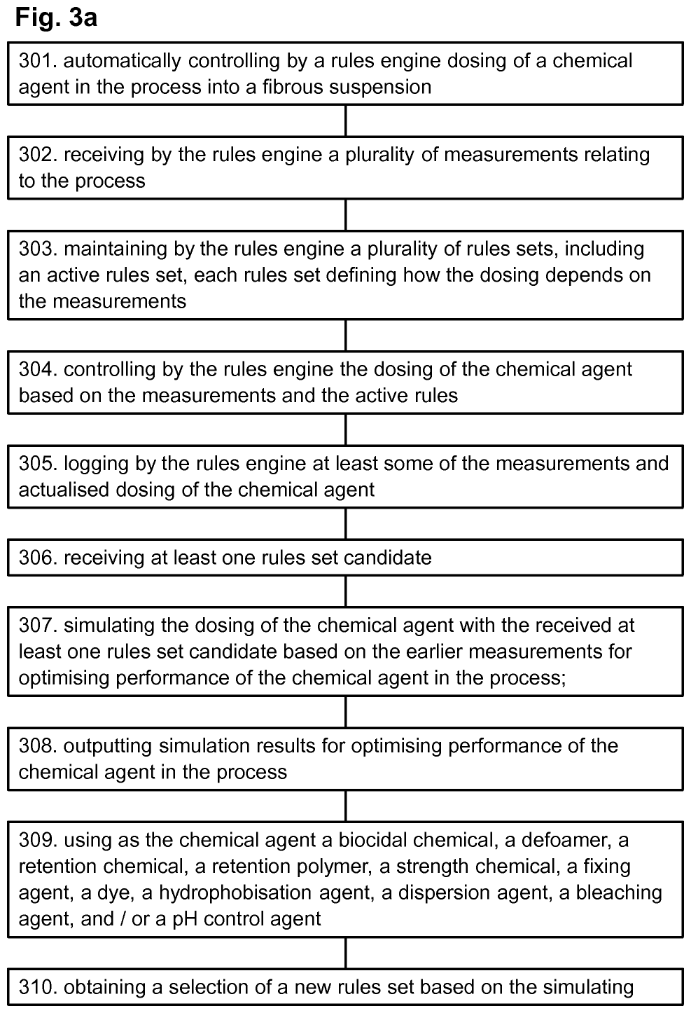

FIG. 2 shows a block diagram of an apparatus 200 according to an example embodiment. The apparatus 200 comprises a communication interface 210; a processor 220; a user interface 230; and a memory 240.

The communication interface 210 comprises in an embodiment a wired and/or wireless communication circuitry, such as Ethernet; Wireless LAN; Bluetooth; GSM; CDMA; WCDMA; LTE; and/or a 5G circuitry. The communication interface can be integrated in the apparatus 200 or provided as a part of an adapter, card, or the like, that is attachable to the apparatus 200. The communication interface 210 may support one or more different communication technologies. The apparatus 200 may also or alternatively comprise more than one of the communication interfaces 210.

In this document, a processor may refer to a central processing unit (CPU); a microprocessor; a digital signal processor (DSP); a graphics processing unit; an application specific integrated circuit (ASIC); a field programmable gate array; a microcontroller; or a combination of such elements.

The user interface may comprise a circuitry for receiving input from a user of the apparatus 200, e.g., via a keyboard; graphical user interface shown on the display of the apparatus 200; speech recognition circuitry; or an accessory device; such as a headset; and for providing output to the user via, e.g., a graphical user interface or a loudspeaker.

The memory 240 comprises a work memory 242 and a persistent memory 244 configured to store computer program code 246 and data 248, such as the rules sets 122 of FIG. 1. The memory 240 may comprise any one or more of: a read-only memory (ROM); a programmable read-only memory (PROM); an erasable programmable read-only memory (EPROM); a random-access memory (RAM); a flash memory; a data disk; an optical storage; a magnetic storage; a smart card; a solid-state drive (SSD); or the like. The apparatus 200 may comprise a plurality of the memories 240. The memory 240 may be constructed as a part of the apparatus 200 or as an attachment to be inserted into a slot;

port; or the like of the apparatus 200 by a user or by another person or by a robot. The memory 240 may serve the sole purpose of storing data or further serve other purposes, such as processing data.

A skilled person appreciates that in addition to the elements shown in FIG. 2, the apparatus 200 may comprise other elements, such as microphones; displays; as well as additional circuitry such as input/output (I/O) circuitry; memory chips; application-specific integrated circuits (ASIC); processing circuitry for specific purposes such as source coding/decoding circuitry; channel coding/decoding circuitry; ciphering/deciphering circuitry; and the like.

In an example embodiment, the apparatus 200 is a computer cloud implemented apparatus. In an example embodiment, the apparatus 200 is a virtual device implemented by cloud computing, one or more computer servers, and/or one or more server clusters.

In an example embodiment, the apparatus 200 comprises or is an Edge device.

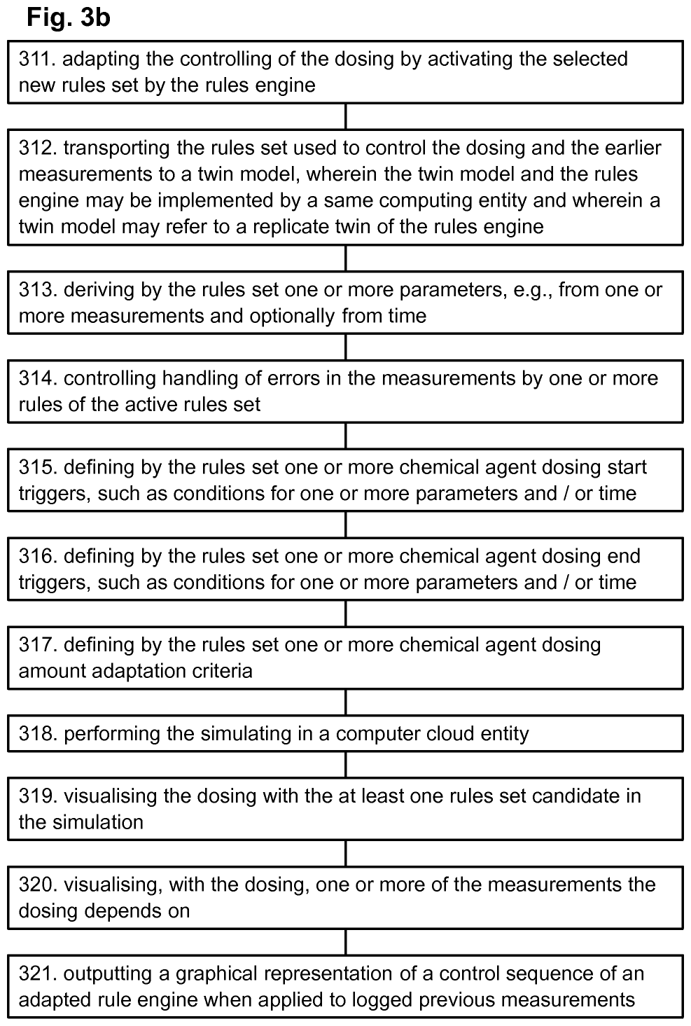

FIGS. 3a and 3b show a flow chart according to an example embodiment illustrating a method for controlling a process in making pulp, paper, or board, comprising various possible steps including some optional steps while also further steps can be included and/or some of the steps can be performed more than once:
301. automatically controlling by a rules engine dosing of a chemical agent in the process into a fibrous suspension;
302. receiving by the rules engine a plurality of measurements relating to the process;
303. maintaining by the rules engine a plurality of rules sets, including an active rules set, each rules set defining how the dosing depends on the measurements;
304. controlling by the rules engine the dosing of the chemical agent based on the measurements and the active rules;
305. logging by the rules engine at least some of the measurements and actualised dosing of the chemical agent;
306. receiving at least one rules set candidate;
307. simulating the dosing of the chemical agent with the received at least one rules set candidate based on the earlier measurements;
308. outputting simulation results for optimising performance of the chemical agent in the process, e.g., graphically or onto a storage medium;
309. using as the chemical agent a biocidal chemical, a defoamer, a retention chemical, a retention polymer, a strength chemical, a fixing agent, a dye, a hydrophobisation agent, a dispersion agent, a bleaching agent, and/or a pH control agent;
310. obtaining a selection of a new rules set based on the simulating;
311. adapting the controlling of the dosing by activating the selected new rules set by the rules engine;
312. transporting the rules set used to control the dosing and the earlier measurements to a twin model, wherein the twin model and the rules engine may be implemented by a same computing entity and wherein a twin model may refer to a replicate twin of the rules engine;
313. deriving by the rules set one or more parameters, e.g., from one or more measurements and optionally from time;
314. controlling handling of errors in the measurements by one or more rules of the active rules set;
315. defining by the rules set one or more chemical agent dosing start triggers, such as conditions for one or more parameters and/or time;
316. defining by the rules set one or more chemical agent dosing end triggers, such as conditions for one or more parameters and/or time;
317. defining by the rules set one or more chemical agent dosing amount adaptation criteria;
318. performing the simulating in a computer cloud entity;
319. visualising the dosing with the at least one rules set candidate in the simulation,
320. visualising, with the dosing, one or more of the measurements the dosing depends on;
321. outputting a graphical representation of a control sequence of an adapted rule engine when applied to logged previous measurements.

A significantly simplified example is next described. The process in question is controlling dosing of a biocide agent into a storage tower of fibrous suspension in making of partially recycled paper. The dosing is made directly to an inflow of a fibrous suspension that comprises partly recycled paper pulp and partly primary fibres. The rules engine controls the dosing initially based on two measurements, inflow rate x₁, and pH x₁, of the suspension after the storage tower. The rule set has following rules:
R1: d=i·x₁ for setting dosing per a quantity unit of incoming fibrous suspension, such as a volume or mass unit, wherein d defines dosing, i defines a suitable basic dosing level per volume;
R2: if x₂<k, then set d: d·(1+j·(k−x₂)); wherein x₂ is pH, j and k are coefficients.

This rules set is based on an assumption that a baseline dosing is needed directly proportionally to the inflow of the suspension, but sometimes changes in the composition of the suspension cause increased biological activity that is indicated by reduction of the pH of the suspension. Hence, the second rule adds dosing when the pH falls below k, such as 6.5, as scaled by term j.

When parameters and actualised dosing are logged, the history data can be used to graphically illustrate how the dosing of the chemical agent has been controlled during the process according to the rules set. However, a need arises to improve the rules set. For example, it may be noticed that with higher surface level (bigger inventory in storage tower), the increased dwell time causes stronger microbial fermentation. Hence, stronger dosing could be added if the surface level exceeds a given threshold. For example, it may also be noticed that some paper quality degradation has happened when the tank has been emptied. A first rules set candidate has rules R1 and R2 as before and adds a rule
R3: if x₃>I, then set d: d·m; wherein x₃ a surface level of the tank, I is a surface level threshold, and m is a dosing boost constant, wherein m is, e.g., 1.15.

A second rules set candidate is defined having rules R1 and R2 and a rule
R4: if x₃>I, then set d: d·(1+m·(x₃−I)).

In the simulation, the two rules set candidates are applied to historical observations (to a selected time period, for example one week) and it is seen how the dosing behavior changes according to the rules set in question. As an output, corresponding graphs can be displayed and an amount of chemical agent consumption and/or other characteristics can be provided to an expert user developing the dosing control. It is then easy to see how and where different rules sets react to different changes in process conditions and how the dosing is adjusted. Here, it is seen how the first rules set candidate triggers a stepwise boosting in the dosing when the surface level exceeds the surface level threshold I. In case the surface level has fluctuated below and above this threshold level in some historical data, the dosing of the first rules set candidate would have changed with a succession of rapid steps. With the second rules set candidate, the simulation results in a gradual change in dosing such that no significant disturbances are likely. A desired rules set is then selected, now in this example the second rules set candidate, and the rules engine is provided with this selection. Then, the active rules set is replaced by the rules engine to adopt the new rules set for in the dosing control to control the manufacturing process. Hence, it can be avoided that dosing is made excessively just to be on the safe side. Instead, the dosing can be targeted to situations where really needed and thus total consumption of chemicals can be minimised and process or product quality stabilised better than before. On reducing margins in the dosing, the accuracy of the dosing control becomes increasingly important. In some example embodiments, this accenting need is addressed by supporting agile verification of new rules set candidates without experimenting with a production process itself.

As the process circumstances may vary widely and in unexpected ways, it is indeed difficult to anticipate all requirements for the rules sets. Sometimes, a good rules set fails in unexpected circumstances. Especially using derived parameters, it may be very difficult to anticipate how a given modification of a rules set would have operated under different circumstances. By using historical data and simulating different rules set candidates with these data, production control can be improved with vastly decreased response times and far more effectively than before. Dosing errors or inaccuracies and resulting process disturbances may be avoided or mitigated.

In an example embodiment, one or more rules of the active rules set control handling of errors in the measurements. For example, one rule can define that the ORP measurement is reliable in a range between −100 mV to +400 mV. The rule can validate measurements in this range. Conversely, if a measurement is −120 mV, then this measurement validation rule defines the measurement as unreliable. While the measurement remains invalid, the rule may replace the measurement value with an error concealment value, such as a predefined safe value. The safe value may be selected from a given point in the middle of the range. In another example, the safe value is determined based on the latest valid values. The safe value may be dynamically defined. For example, if the measurement has decreased to and beyond a valid range endpoint, then the safe value may be dynamically defined as a value that is a nearest value to the measurement and resides withing the valid range.

Other or additional measurements could also be used in other rules set candidates, such as conductivity in conjunction with surface level or time elapsed since last washing of the storage tower.

In an example embodiment, the active rules set is monitored for efficiency. For example, actually implemented dosing of chemicals may be logged. Monitored data may be compared with one or more fall-back criteria for triggering a fall-back action in which a fall-back rules set may be adopted at least temporarily instead of the active rules set when the active rules set does not appear to function properly. The efficiency may refer to that how well the active rules set works in the process control, e.g., as a measure of chemical consumption and/or some quality measurements.

The fall-back criteria may be at least partially or entirely global, applicable to all rules sets. Alternatively, or additionally, there may be fall-back criteria defined within one or more rules sets. The fall-back criteria defined in a rules set may prevail over global fall-back rules.

The fall-back criteria may define one or more comparisons for the monitored data, such as a given term (or parameter) exceeding, meeting, or falling below, of a limit. The term may be defined based on one or more different measurements. The term may be defined based on real-time data. The term may be defined based on data accrued over a comparison period that extends over at least 10 min, 30 min, 1 hour, 4 hours, 12 hours, 24 hours, 48 hours, or 168 hours. The term may be or comprise an average, mean, standard deviation, variance, sum, or any combination thereof. For example, the term may be an average concentration of a chemical in a suspension during last 6 hours, or an amount of chemical dosing, or a sum of any (derived) measurements, during the comparison period. The comparison period may be an ongoing day, week, or other recurring period.

The fall-back criteria may be defined for each of a plurality of simultaneously used rules sets, e.g., in case that there is a plurality of different process control systems each using its own rules set.

The fall-back rules set may be defined for each of a plurality of simultaneously used rules sets, e.g., in case that there is a plurality of different process control systems each using its own rules set.

The fall-back rules may define a fall-back action such as stopping of chemical dosing or decreasing the chemical dosing for a given correction period. The fall-back action may include issuing an alarm to an operator. The correction period may last for at least 10 min, 30 min, 1 hour, 4 hours, 12 hours, or 24 hours.

The fall-back criteria may comprise one or more criteria for a stability of a process.

Any of the afore described methods, method steps, or combinations thereof, may be controlled or performed using hardware; software; firmware; or any combination thereof.

The software and/or hardware may be local; distributed; centralised; virtualised; or any combination thereof. Moreover, any form of computing, including computational intelligence, may be used for controlling or performing any of the afore described methods, method steps, or combinations thereof. Computational intelligence may refer to, for example, any of artificial intelligence; neural networks; fuzzy logics; machine learning; genetic algorithms; evolutionary computation; or any combination thereof.

As a technical effect, the dosing of the chemical agent may be optimised using measurement history relating to the process with simulated rule engines such that responses of different rule engines can be instantaneously determined for optimising the performance of the chemical agent. The simulating may comprise computing one or more estimated properties for the end product.

Various embodiments have been presented. It should be appreciated that in this document, words comprise; include; and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A method for controlling a process in making pulp, paper, or board, comprising:
automatically controlling by a rules engine a dosing of a chemical agent in the process into a fibrous suspension;
receiving by the rules engine a plurality of measurements relating to the process;
maintaining by the rules engine a plurality of rules sets, including an active rules set, each rules set defining how the dosing depends on the measurements;
performing the controlling by the rules engine of the dosing of the chemical agent based on the measurements and the active rules set;
logging by the rules engine at least some of the measurements and actualised dosing of the chemical agent;
receiving at least one rules set candidate;
simulating the dosing of the chemical agent with the received at least one rules set candidate based on the earlier measurements;
outputting simulation results for optimising performance of the chemical agent in the process;
obtaining a selection of a new rules set based on the simulating; and
causing adapting of the controlling of the dosing by activating the selected new rules set by the rules engine.

2. The method of claim 1, wherein the rules set used to control the dosing and the earlier measurements are transported to a twin model.

3. The method of claim 1, wherein the rules set is configured to derive one or more parameters from one or more measurements and optionally from time.

4. The method of claim 1, further comprising controlling handling of errors in the measurements by one or more rules of the active rules set.

5. The method of claim 1, further comprising performing the simulating by the rules engine.

6. The method of claim 1, further comprising defining by the rules set one or more chemical agent dosing start triggers.

7. The method of claim 1, further comprising defining by the rules set one or more chemical agent dosing end triggers.

8. The method of claim 1, comprising performing the simulating in a computer cloud entity.

9. The method of claim 1, further comprising outputting a graphical representation of a control sequence of an adapted rule engine when applied to logged previous measurements.

10. The method of claim 1, wherein the suspension comprises cellulose and hemicellulose, and has at least 0.5, 1, 2, 5, 10 or 15 weight percent of dry matter.

11. The method of claim 1, wherein the suspension comprises cellulose and hemicellulose, and the suspension has at most 1, 2, 5, 10 or 20 weight percent of dry matter.

12. The method of claim 1, wherein the chemical agent comprises a biocidal chemical, a defoamer, a retention chemical, a retention polymer, a strength chemical, a fixing agent, a dye, a hydrophobisation agent, a dispersion agent, a bleaching agent, and/or a pH control agent.

13. The method of claim 1, further comprising
monitoring efficiency of the active rules set;
determining triggering of fall-back criteria; and
taking a fall-back action on determining the triggering of the fall-back criteria, instead of continuing to control the process according to the active rules set.

14. An apparatus comprising means for performing the method of claim 1.

15. A non-transitory computer readable medium having stored there on a computer program comprising computer executable program code configured, when executed, to cause a computer to perform the method of claim 1.
